(19) United States
(12) Patent Application Publication
(10) Pub. No.: US 2014/0275295 A1
(43) Pub. Date: Sep. 18, 2014

(54) METHOD FOR TREATING TYPE I AND TYPE II DIABETES

(71) Applicants: Hiroyuki Kouji, Kanagawa (JP); Takenao Odagami, Kanagawa (JP)

(72) Inventors: Hiroyuki Kouji, Kanagawa (JP); Takenao Odagami, Kanagawa (JP)

(21) Appl. No.: 14/352,439

(22) PCT Filed: Oct. 19, 2012
(86) PCT No.: PCT/IB2012/003032

§ 371 Date: Apr. 17, 2014

Related U.S. Application Data
(60) Provisional application No. 61/549,121, filed on Oct. 19, 2011.

(51) Int. Cl. A61K 45/00

(52) U.S. Cl. CPC A61K 45/00
USPC 514/789

(57) ABSTRACT

The present disclosure relates generally to alpha-helix mimetic structures and specifically to alpha-helix mimetic structures that are inhibitors of β-catenin. The disclosure also relates to applications in the treatment of diabetes and diabetic conditions such as diabetic neuropathy, and pharmaceutical compositions comprising such alpha helix mimetic β-catenin inhibitors.

### BACKGROUND OF THE DISCLOSURE

The Wnt gene family encodes a large class of secreted proteins related to the Int1/Wnt1 proto-oncogene and Drosophila wingless (“Wg”), a Drosophila Wnt1 homologue (Cadigan et al. (1997) Genes & Development 11:3286-3305). Wnts are expressed in a variety of tissues and organs and are required for many developmental processes, including segmentation in Drosophila; endoderm development in C. elegans; and establishment of limb polarity, neural crest differentiation, kidney morphogenesis, sex determination, and brain development in mammals (Parr, et al. (1994) Curr. Opinion Genetics & Devel. 4:523-528). The Wnt pathway is a master regulator in development, both during embryogenesis and in the mature organism (Eastman, et al. (1999) Curr Opin Cell Biol 11: 233-240; Peifer, et al. (2000) Science 287: 1606-1609).

Wnt signals are transduced by the Frizzled (“Fz”) family of seven transmembrane domain receptors (Bhanot et al. (1996) Nature 382:225-230). Frizzled cell-surface receptors (Fzd) play an essential role in both canonical and non-canonical Wnt signaling. In the canonical pathway, upon activation of Fzd and LRP5/6 (low-density-lipoprotein receptor-related protein 5 and 6) by Wnt proteins, a signal is generated that prevents the phosphorylation and degradation of β-catenin by the “β-catenin destruction complex,” permitting stable β-catenin translocation and accumulation in the nucleus, and therefore Wnt signal transduction. (Perrimon (1994) Cell 76:781-784) (Miller, J. R. (2001) Genome Biology; 3(1):1-15). The non-canonical Wnt signaling pathway is less well defined: there are at least two non-canonical Wnt signaling pathways that have been proposed, including the planar cell polarity (PCP) pathway, the Wnt/Ca++ pathway, and the convergence extension pathway.

Glycogen synthase kinase 3 (GSK3), the tumor suppressor gene product APC (adenomatous polyposis coli) (Gumbiner (1997) Curr. Biol. 7:R443-436), and the scaffolding protein Axin, are all negative regulators of the Wnt pathway, and together form the “β-catenin destruction complex.” In the absence of a Wnt ligand, these proteins form a complex and promote phosphorylation and degradation of β-catenin, whereas Wnt signaling inactivates the complex and prevents β-catenin degradation. Stabilized β-catenin translocates to the nucleus as a result, where it binds TCF (T cell factor) transcription factors (also known as lymphoid enhancer-binding factor-1 (LEF1)) and serves as a coactivator of TCF/LEF-induced transcription (Bienz, et al. (2000) Cell 103: 311-320; Polakis, et al. (2000) Genes Dev 14: 1837-1851).

Wnt signaling occurs via canonical and non-canonical mechanisms. In the canonical pathway, upon activation of Fzd and LRP5/6 by Wnt proteins, stabilized β-catenin accumulates in the nucleus and leads to activation of TCF target genes (as described above; Miller, J. R. (2001) Genome Biology; 3(1):1-15). The non-canonical Wnt signaling pathway is less well defined: at least two non-canonical Wnt signaling pathways have been proposed, including the planar cell polarity (PCP) pathway and the Wnt/Ca⁺⁺ pathway.

Disorders associated with pathologically high or low levels of Wnt signaling include, but are not limited to, osteoporosis, osteoarthritis, polycystic kidney disease, diabetes, schizophrenia, vascular disease, cardiac disease, non-oncogenic proliferative diseases, and neurodegenerative diseases such as Alzheimer's disease.

Diabetes Mellitus describes a metabolic disorder characterized by chronic hyperglycemia with disturbances of carbohydrate, fat and protein metabolism that result from defects in insulin secretion, insulin action, or both. The effects of Diabetes Mellitus include long-term damage, dysfunction and failure of various organs. Diabetes can be present with characteristic symptoms such as thirst, polyuria, blurring of vision, chronic infections, slow wound healing, and weight loss. In its most severe forms, ketoacidosis or a non-ketotic hyperosmolar state can develop and lead to stupor, coma and, in the absence of effective treatment, death. Often symptoms are not severe, not recognized, or can be absent. Consequently, hyperglycemia sufficient to cause pathological and functional changes can be present for a long time, occasionally up to ten years, before a diagnosis is made, usually by the detection of high levels of glucose in urine after overnight fasting during a routine medical work-up. The long-term effects of Diabetes Mellitus include progressive development of complications such as retinopathy with potential blindness, nephropathy that can lead to renal failure, neuropathy, microvascular changes, and autonomic dysfunction. People with Diabetes are also at increased risk of cardiovascular, peripheral vascular, and cerebrovascular disease (together, “arteriovascular” disease). There is also an increased risk of cancer. Several pathogenetic processes are involved in the development of Diabetes. These include processes which destroy the insulin-secreting beta cells of the pancreas with consequent insulin deficiency, and changes in liver and smooth muscle cells that result in the resistance to insulin uptake. The abnormalities of carbohydrate, fat and protein metabolism are due to deficient action of insulin on target tissues resulting from insensitivity to insulin or lack of insulin.

Regardless of the underlying cause, Diabetes Mellitus is subdivided into Type 1 Diabetes and Type 2 Diabetes. Type 1 Diabetes results from autoimmune mediated destruction of the beta cells of the pancreas. The rate of destruction is variable, and the rapidly progressive form is commonly observed in children, but can also occur in adults. The slowly progressive form of Type 1 Diabetes generally occurs in adults and is sometimes referred to as latent autoimmune Diabetes in adults (LADA). Some patients, particularly children and adolescents, can exhibit ketoacidosis as the first manifestation of the disease. Others have modest fasting hyperglycemia that can rapidly change to severe hyperglycemia and/or ketoacidosis in the presence of infection or other stress. Still others, particularly adults, can retain residual beta cell function sufficient to prevent ketoacidosis for many years. Individuals with this form of Type 1 Diabetes often become dependent on insulin for survival and are at risk for ketoacidosis. Patients with Type 1 Diabetes exhibit little or no insulin secretion as manifested by low or undetectable levels of plasma C-peptide. However, there are some forms of Type 1 Diabetes which have no known etiology, and some of these patients have permanent insulinopenia and are prone to ketoacidosis, but have no evidence of autoimmunity. These patients are referred to as “Type 1 idiopathic.”

Type 2 Diabetes is the most common form of Diabetes and is characterized by disorders of insulin action and insulin secretion, either of which can be the predominant feature. Both are usually present at the time that this form of Diabetes is clinically manifested. Type 2 Diabetes patients are characterized with a relative, rather than absolute, insulin deficiency and are resistant to the action of insulin. At least initially, and often throughout their lifetime, these individuals do not need insulin treatment to survive. Type 2 Diabetes accounts for 90-95% of all cases of Diabetes. This form of Diabetes can go undiagnosed for many years because the hyperglycemia is often not severe enough to provoke noticeable symptoms of Diabetes or symptoms are simply not recognized. The majority of patients with Type 2 Diabetes are obese, and obesity itself can cause or aggravate insulin resistance. Many of those who are not obese by traditional weight criteria can have an increased percentage of body fat distributed predominantly in the abdominal region (visceral fat). Ketoacidosis is infrequent in this type of Diabetes and usually arises in association with the stress of another illness. Whereas patients with this form of Diabetes can have insulin levels that appear normal or elevated, the high blood glucose levels in these diabetic patients would be expected to result in even higher insulin values had their beta cell function been normal. Thus, insulin secretion is often defective and insufficient to compensate for the insulin resistance. On the other hand, some hyperglycemic individuals have essentially normal insulin action, but markedly impaired insulin secretion.

Diabetic retinopathy is an eye disease that develops in diabetes due to changes in the cells that line blood vessels. When glucose levels are high, as in diabetes, glucose can cause damage in a number of ways. For example, glucose, or a metabolite of glucose, binds to the amino groups of proteins, leading to tissue damage. In addition, excess glucose enters the polyol pathway resulting in accumulations of sorbitol. Sorbitol cannot be metabolized by the cells of the retina and can contribute to high intracellular osmotic pressure, intracellular edema, impaired diffusion, tissue hypoxia, capillary cell damage, and capillary weakening. Diabetic retinopathy involves thickening of capillary basement membranes and prevents pericytes from contacting endothelial cells of the capillaries. Loss of pericytes increases leakage of the capillaries and leads to breakdown of the blood-retina barrier. Weakened capillaries lead to aneurysm formation and further leakage. These effects of hyperglycemia can also impair neuronal functions in the retina. This is an early stage of diabetic retinopathy termed nonproliferative diabetic retinopathy.

Retinal capillaries can become occluded in diabetes causing areas of ischemia in the retina. The non-perfused tissue responds by eliciting new vessel growth from existing vessels (angiogenesis). These new blood vessels can also cause loss of sight.

Given the difficulty in maintaining good glycemic control in human diabetics, development of drugs that inhibit or slow retinal capillary cell and retinal neuron damage would provide a means of reducing the early cellular damage that occurs in diabetic retinopathy.

There is an urgent need for new treatments for diabetes and diabetic complications, such as diabetic retinopathy.

### BRIEF SUMMARY OF THE DISCLOSURE

The present disclosure relates generally to alpha-helix mimetic structures and specifically to alpha-helix mimetic structures that are inhibitors of β-catenin. The disclosure also relates to applications in the treatment of diabetes and diabetic conditions such as diabetic neuropathy, and pharmaceutical compositions comprising such alpha helix mimetic β-catenin inhibitors.

### DETAILED DESCRIPTION OF THE DISCLOSURE

Recently, non-peptide compounds have been developed which mimic the secondary structure of reverse-turns found in biologically active proteins or peptides. For example, U.S. Pat. No. 5,440,013 and published PCT Applications Nos. WO94/03494, WO01/00210A1, and WO01/16135A2 each disclose conformationally constrained, non-peptidic compounds, which mimic the three-dimensional structure of reverse-turns. In addition, U.S. Pat. No. 5,929,237 and its continuation-in-part U.S. Pat. No. 6,013,458, disclose conformationally constrained compounds which mimic the secondary structure of reverse-turn regions of biologically active peptides and proteins. In relation to reverse-turn mimetics, conformationally constrained compounds have been disclosed which mimic the secondary structure of alpha-helix regions of biologically active peptide and proteins in WO2007/056513 and WO2007/056593.

Disclosed herein are treatments and compounds for treatment of Type I and Type II Diabetes Mellitus, diabetic retinopathy, and gestational diabetes.

The structures and compounds of the alpha helix mimetic β-catenin inhibitors of this invention are disclosed in WO 2010/044485, WO 2010/128685, WO 2009/148192, and US 2011/0092459, each of which is incorporated herein by reference in its entirety. These compounds have now been found to be useful in the treatment of diabetes, diabetic retinopathy, and gestational diabetes. While not wishing to be bound, the effectiveness of these compounds in treating these conditions is based in part on the ability of these compounds to block TCF4/β-catenin transcriptional pathway by inhibiting CBP, thus altering wnt pathway signaling, which has been found to improve diabetic outcomes.

A “β-catenin inhibitor” is a substance that can reduce or prevent β-catenin activity. β-catenin activities include translocation to the nucleus, binding with TCF (T cell factor) transcription factors, and coactivating TCF transcription factor-induced transcription of TCF target genes.

Disclosed herein are alpha helix mimetic β-catenin inhibitor compounds for treatment of diabetic conditions.

The term “diabetic conditions” as used herein includes both insulin-dependent diabetes (also known as IDDM, type-1 diabetes), and non-insulin-independent diabetes (also known as NIDDM, type-2 diabetes), as well as pre-diabetes and gestational diabetes. “Diabetic retinopathy” is an ophthalmic diabetic condition.

As used herein, “treatment” refers to clinical intervention in an attempt to alter the disease course of the individual or cell being treated, and can be performed during the course of clinical pathology. Therapeutic effects of treatment include without limitation, preventing recurrence of disease, alleviation of symptoms, diminishment of any direct or indirect pathological consequences of the disease, decreasing the rate of disease progression, amelioration or palliation of the disease state, and remission or improved prognosis.

As used herein, the terms “therapeutically effective amount” and “effective amount” are used interchangeably to refer to an amount of a composition of the invention that is sufficient to result in the prevention of the development or onset of diabetes, or one or more symptoms thereof, to enhance or improve the effect(s) of another therapy, and/or to ameliorate one or more symptoms of diabetes. For a diabetic patient, a preferred therapeutically effective amount is an amount effective to control or normalize blood sugar levels.

A therapeutically effective amount can be administered to a patient in one or more doses sufficient to palliate, ameliorate, stabilize, reverse or slow the progression of the disease, or otherwise reduce the pathological consequences of the disease, or reduce the symptoms of the disease. The amelioration or reduction need not be permanent, but can be for a period of time ranging from at least one hour, at least one day, or at least one week or more. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition, as well as the route of administration, dosage form and regimen and the desired result.

As used herein, the terms “subject” and “patient” are used interchangeably and refer to an animal, preferably a mammal such as a non-primate (e.g., cows, pigs, horses, cats, dogs, rats etc.) and a primate (e.g., monkey and human), and most preferably a human.

The alpha helix mimetic β-catenin inhibitors described herein can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

The alpha helix mimetic β-catenin inhibitors described herein are useful to prevent or treat disease. Specifically, the disclosure provides for both prophylactic and therapeutic methods of treating a subject at risk of (or susceptible to) diabetes and diabetic conditions. Accordingly, the present methods provide for the prevention and/or treatment of diabetes and diabetic conditions in a subject by administering an effective amount of an alpha helix mimetic β-catenin inhibitor to a subject in need thereof. For example, a subject can be administered the alpha helix mimetic β-catenin inhibitors in an effort to improve one or more of the factors of a diabetic condition.

In an aspect the present invention provides a pharmaceutical composition or combination comprising (a) an alpha helix mimetic β-catenin inhibitor as described herein, and (b) an antidiabetic agent or a pharmaceutically acceptable salt thereof.

Classes of antidiabetic agents include, but are not limited to, the biguanide class, thiazolidindione class, sulfonylurea class, glinide class, alpha-glucosidase inhibitor class, and GLP-1 analogue class.

The antidiabetic agent is selected from the group G3 which includes biguanides, thiazolidindiones, sulfonylureas, glinides, inhibitors of alpha-glucosidase, GLP-1 analogues or a pharmaceutically acceptable salt thereof.

The group G3 comprises biguanides. Examples of biguanides are metformin, phenformin and buformin. A preferred biguanide is metformin. An alpha helix mimetic β-catenin inhibitor in combination with a biguanide, in particular metformin, can provide more efficacious glycemic control and/or can act together with the biguanide, for example to reduce weight, that has e.g. overall beneficial effects on the metabolic syndrome which is commonly associated with type 2 diabetes mellitus.

The term “metformin” as employed herein refers to metformin or a pharmaceutically acceptable salt thereof such as the hydrochloride salt, the metformin (2:1) fumarate salt, and the metformin (2:1) succinate salt, the hydrobromide salt, the p-chlorophenoxy acetate or the embonate, and other known metformin salts of mono and dibasic carboxylic acids. It is preferred that the metformin employed herein is the metformin hydrochloride salt.

The group G3 comprises thiazolidindiones. Examples of thiazolidindiones (TZDs) are pioglitazone and rosiglitazone. TZD therapy is associated with weight gain and fat redistribution. In addition, TZDs cause fluid retention and are not indicated in patients with congestive heart failure. Long term treatment with TZDs is further associated with an increased risk of bone fractures. An alpha helix mimetic β-catenin inhibitor in combination with a thiazolidindione, in particular pioglitazone, can provide more efficacious glycemic control and/or can minimize side effects of the treatment with TZD.

The group G3 comprises sulfonylureas. Examples of sulfonylureas are glibenclamide, tolbutamide, glimepiride, glipizide, gliquidone, glibornuride, glyburide, glisoxepide and gliclazide. Preferred sulfonylureas are tolbutamide, gliquidone, glibenclamide and glimepiride, in particular glibenclamide and glimepiride. As the efficacy of sulfonylureas wears off over the course of treatment, a combination of an alpha helix mimetic β-catenin inhibitor with a sulfonylurea can offer additional benefit to the patient in terms of better glycemic control. Also, treatment with sulfonylureas is normally associated with gradual weight gain over the course of treatment and an alpha helix mimetic β-catenin inhibitor can minimize this side effect and/or improve the metabolic syndrome. Also, an alpha helix mimetic β-catenin inhibitor in combination with a sulfonylurea can minimize hypoglycemia which is another undesirable side effect of sulfonylureas. This combination can also allow a reduction in the dose of sulfonylureas, which can also translate into less hypoglycemia.

Each term of the group “glibenclamide”, “glimepiride”, “gliquidone”, “glibornuride”, “gliclazide”, “glisoxepide”, “tolbutamide” and “glipizide” as employed herein refers to the respective active drug or a pharmaceutically acceptable salt thereof.

The group G3 comprises glinides. Examples of glinides are nateglinide, repaglinide and mitiglinide. As their efficacy wears off over the course of treatment, a combination of an alpha helix mimetic β-catenin inhibitor with a meglitinide can offer additional benefit to the patient in terms of better glycemic control. Also, treatment with meglitinides is normally associated with gradual weight gain over the course of treatment and an alpha helix mimetic β-catenin inhibitor can minimize this side effect of the treatment with an meglitinide and/or improve the metabolic syndrome. Also, an alpha helix mimetic β-catenin inhibitor in combination with a meglitinide can minimize hypoglycemia which is another undesirable side effect of meglitinides. This combination can also allow a reduction in the dose of meglitinides, which can also translate into less hypoglycemia.

The group G3 comprises inhibitors of alpha-glucosidase. Examples of inhibitors of alpha-glucosidase are acarbose, voglibose and miglitol. Additional benefits from the combination of an alpha helix mimetic β-catenin inhibitor and an alpha-glucosidase inhibitor can relate to more efficacious glycemic control, e.g. at lower doses of the individual drugs, and/or reducement of undesirable gastrointestinal side effects of alpha-glucosidase inhibitors.

Each term of the group “acarbose”, “voglibose” and “miglitol” as employed herein refers to the respective active drug or a pharmaceutically acceptable salt thereof.

The group G3 comprises inhibitors of GLP-1 analogues. Examples of GLP-1 analogues are exenatide, liraglutide, taspoglutide, semaglutide, albiglutide, and lixisenatide. The combination of an alpha helix mimetic β-catenin inhibitor and a GLP-1 analogue can achieve a superior glycemic control, e.g. at lower doses of the individual drugs. In addition, the body weight reducing capability of the GLP-1 analogue can be positively act together with the properties of the alpha helix mimetic β-catenin inhibitor. On the other hand, a reduction of side effects (e.g. nausea, gastrointestinal side effects like vomiting) can be obtained when a reduced dose of the GLP-1 analogue is applied in the combination with an alpha helix mimetic β-catenin inhibitor.

Each term of the group “exenatide”, “liraglutide”, “taspoglutide”, “semaglutide”, “albiglutide” and “lixisenatide” as employed herein refers to the respective active drug or a pharmaceutically acceptable salt thereof.

According to another aspect of the invention, there is provided a method for preventing, slowing the progression of, delaying or treating a metabolic disorder selected from the group consisting of type 1 diabetes mellitus, type 2 diabetes mellitus, impaired glucose tolerance (IGT), impaired fasting blood glucose (IFG), hyperglycemia, postprandial hyperglycemia, overweight, obesity and metabolic syndrome in a patient in need thereof characterized in that an alpha helix mimetic β-catenin inhibitors described herein and, optionally, a second and, optionally, a third antidiabetic agent as defined hereinbefore and hereinafter are administered, for example in combination, to the patient.

As by the use of a pharmaceutical composition or combination according to this invention, an improvement of the glycemic control in patients in need thereof is obtainable, also those conditions and/or diseases related to or caused by an increased blood glucose level can be treated.

Diabetes is characterized by a fasting plasma glucose level of greater than or equal to 126 mg/dl. A diabetic subject has a fasting plasma glucose level of greater than or equal to 126 mg/dl. Prediabetes is characterized by an impaired fasting plasma glucose (FPG) level of greater than or equal to 110 mg/dl and less than 126 mg/dl; or impaired glucose tolerance; or insulin resistance. A prediabetic subject is a subject with impaired fasting glucose (a fasting plasma glucose (FPG) level of greater than or equal to 110 mg/dl and less than 126 mg/dl); or impaired glucose tolerance (a 2 hour plasma glucose level of >140 mg/dl and <200 mg/dl); or insulin resistance, resulting in an increased risk of developing diabetes.

Retinopathy is a leading cause of blindness in type I diabetes, and is also common in type II diabetes. The degree of retinopathy depends on the duration of diabetes, and generally begins to occur ten or more years after onset of diabetes. Diabetic retinopathy can be classified as non-proliferative, where the retinopathy is characterized by increased capillary permeability, edema and exudates, or proliferative, where the retinopathy is characterized by neovascularisation extending from the retina to the vitreous, scarring, deposit of fibrous tissue and the potential for retinal detachment. Diabetic retinopathy is believed to be caused by the development of glycosylated proteins due to high blood glucose.

“Gestational diabetes mellitus” refers to any degree of glucose intolerance with onset or first recognition during pregnancy. Clinical diagnosis is generally based on a multi-step process. The evaluation is most typically performed by measuring plasma glucose 1 hour after a 50-gram oral glucose challenge test in either the fasted or the unfasted state. If the value in the glucose challenge test is ≦140 mg/dl, a 3-hr 100 g oral glucose tolerance test is done. If two or more of the following criteria are met, the patient is considered in need of glycemic control: fasted venous plasma ≦105 mg/dl; venous plasma ≦190 ma/dl at 1 hr, venous plasma ≦165 mg/dl at 2 hr or venous plasma ≦145 mg/dl at 3 hr. Williams et al., Diabetes Care 22: 418-421, 1999.

The compounds (the above-mentioned alpha helix mimetic β-catenin inhibitors) and compositions described herein are useful for treatment of diabetic conditions including type 1 diabetes and type 2 diabetes. The compounds and compositions described herein are also useful for treatment and/or prevention of pre-diabetes and/or gestational diabetes mellitus.

Treatment of diabetes mellitus refers to the administration of a compound or combination described herein to treat a diabetic subject. One outcome of the treatment of diabetes is to reduce an increased plasma glucose concentration. Another outcome of the treatment of diabetes is to reduce an increased insulin concentration. Still another outcome of the treatment of diabetes is to reduce an increased blood triglyceride concentration. Still another outcome of the treatment of diabetes is to increase insulin sensitivity. Still another outcome of the treatment of diabetes can be enhancing glucose tolerance in a subject with glucose intolerance. Still another outcome of the treatment of diabetes is to reduce insulin resistance. Another outcome of the treatment of diabetes is to normalize plasma insulin levels. Still another outcome of treatment of diabetes is an improvement in glycemic control, particularly in type 2 diabetes. Yet another outcome of treatment is to increase hepatic insulin sensitivity.

An alpha helix mimetic β-catenin inhibitor can also be administered to a subject to treat or prevent diabetic retinopathy. Diabetic retinopathy is characterized by capillary microaneurysms and dot hemorrhaging. Thereafter, microvascular obstructions cause cotton wool patches to form on the retina. Moreover, retinal edema and/or hard exudates can form in individuals with diabetic retinopathy due to increased vascular hyperpermeability. Subsequently, neovascularization appears and retinal detachment is caused by traction of the connective tissue grown in the vitreous body. Iris rubeosis and neovascular glaucoma can also occur which, in turn, can lead to blindness. The symptoms of diabetic retinopathy include, but are not limited to, difficulty reading, blurred vision, sudden loss of vision in one eye, seeing rings around lights, seeing dark spots, and/or seeing flashing lights.

Any suitable route of administration can be employed for providing a mammal, especially a human, with an effective dose of a compound described herein. For example, oral, rectal, topical, parenteral, ocular, pulmonary, nasal, and the like can be employed. Dosage forms include tablets, troches, dispersions, suspensions, solutions, capsules, creams, ointments, aerosols, and the like. Preferably compounds described herein are administered orally.

The effective dosage of active ingredient employed can vary depending on the particular compound employed, the mode of administration, the condition being treated and the severity of the condition being treated. Such dosage can be ascertained readily by a person skilled in the art.

When treating or controlling diabetes mellitus and/or other diseases for which compounds described herein are indicated, generally satisfactory results are obtained when the compounds described herein are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams. In the case of a 70 kg adult human, the total daily dose will generally be from about 1 milligram to about 500 milligrams. For a particularly potent compound, the dosage for an adult human can be as low as 0.1 mg. In some cases, the daily dose can be as high as 1 gram. The dosage regimen can be adjusted within this range or even outside of this range to provide the optimal therapeutic response.

Oral administration will usually be carried out using tablets or capsules. Examples of doses in tablets and capsules are 0.1 mg, 0.25 mg, 0.5 mg, 1 mg, 2 mg, 5 mg, 10 mg, 15 mg, 20 mg, 25 mg, 30 mg, 40 mg, 50 mg, 100 mg, 200 mg, 250 mg, 300 mg, 400 mg, 500 mg, and 750 mg. Other oral forms can also have the same or similar dosages.

Also described herein are pharmaceutical compositions which comprise a compound described herein and a pharmaceutically acceptable carrier. The pharmaceutical compositions described herein comprise a compound described herein or a pharmaceutically acceptable salt as an active ingredient, as well as a pharmaceutically acceptable carrier and optionally other therapeutic ingredients. A pharmaceutical composition can also comprise a prodrug, or a pharmaceutically acceptable salt thereof, if a prodrug is administered.

The compositions can be suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They can be conveniently presented in unit dosage form and prepared by any of the methods well-known in the art of pharmacy.

In practical use, the compounds described herein can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). In preparing the compositions as oral dosage form, any of the usual pharmaceutical media can be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are employed. If desired, tablets can be coated by standard aqueous or nonaqueous techniques. Such compositions and preparations should contain at least 0.1 percent of active compound. The percentage of active compound in these compositions may, of course, be varied and can conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like can also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it can contain, in addition to materials of the above type, a liquid carrier such as a fatty oil.

Various other materials can be present as coatings or to modify the physical form of the dosage unit. For instance, tablets can be coated with shellac, sugar or both. A syrup or elixir can contain, in addition to the active ingredient, sucrose as a sweetening agent, methyl and propylparabens as preservatives, a dye and a flavoring such as cherry or orange flavor.

For ophthalmic applications, the therapeutic compound is formulated into solutions, suspensions, and ointments appropriate for use in the eye. For ophthalmic formulations generally, see Mitra (ed.), Ophthalmic Drug Delivery Systems, Marcel Dekker, Inc., New York, N.Y. (1993) and also Havener, W. H., Ocular Pharmacology, C.V. Mosby Co., St. Louis (1983). Ophthalmic pharmaceutical compositions can be adapted for topical administration to the eye in the form of solutions, suspensions, ointments, creams or as a solid insert. For a single dose, from between 0.1 ng to 5000 .mu.g, 1 ng to 500 .mu.g, or 10 ng to 100 .mu.g of the aromatic-cationic peptides can be applied to the human eye.

The ophthalmic preparation can contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

The ophthalmic solution or suspension can be administered as often as necessary to maintain an acceptable level of the alpha helix mimetic β-catenin inhibitor in the eye. Administration to the mammalian eye can be about once or twice daily.

Compounds described herein can also be administered parenterally. Solutions or suspensions of these active compounds can be prepared in water suitably mixed with a surfactant or mixture of surfactants such as hydroxypropylcellulose, polysorbate 80, and mono and diglycerides of medium and long chain fatty acids. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

### EXAMPLES

### Example 1

### Reporter Gene Assay

Screening for inhibitory action of the Wnt signaling pathway was carried out according to the following procedure using the stably transfected cell line Hek-293, STF1.1
Growth Medium: DMEM, 10% FBS, Pen-Strep, supplemented with 400 g/mL G418 to maintain selection of SuperTOPFLASH driven Luciferase gene
1. On the day prior to assay, cells were split into a white opaque 96-well plate at 20,000 cells per well in 200 microliters of complete growth medium.
2. Plate was incubated overnight at 37° C., 5% CO₂ to allow the cells to attach to the surface
3. The next day, the inhibitors were prepared to be tested in complete growth medium, without G418, at 2× the desired final concentration (all conditions are done in duplicates)
4. The existing medium was carefully pipetted from each well using a multiple pipettor.
5. 50 microliters of fresh growth medium (without G148) containing the inhibitor (Compound A) was added to each well.
6. 2 wells contained medium only, 2 wells were used for a stimulation control, 2 wells were used for DMSO control, and wells were used for the positive control ICG-001 (2, 5, and 10 micromolar).
7. Once all inhibitors and controls are added, the plate was incubated for 1 hour at 37° C., 5% CO₂.
8. While the plate was incubated, fresh 20 mM LiCl in complete growth medium (without G418) was prepared.
9. After 1 hour, the plate was removed from the incubator and 50 microliters of the medium containing 20 mM LiCl was added to each well, except for the two wells of the unstimulated control (to which were added 50 microliters of complete medium alone).
10. The plate was incubated for 24 hours at 37° C., 5% CO₂.
11. After 24 hours, 100 microliters of BrightGlo (Promega, Cat. #: G7573) was added to each well.
12. The plate was shaken for 5 minutes to ensure complete lysis.
13. The plate was read using a Packard TopCount.

It was found that the compound A had an inhibitory activity more than 50% at the concentration of 1 microM (μM) as determined by reporter gene assay.

### Example 2

### Blood Glucose Level

Patients with cancer and with type II diabetes were enrolled in the clinical trial (Safety and Efficacy Study in Subjects With Advanced Solid) as shown below. The efficacy of an alpha helix mimetic β-catenin inhibitor compound (Compound A) for the treatment of diabetes was investigated by measurement of blood glucose level of the patients. Conditions of patients enrolled in this study are as follows:

(Patients 1) Male 66 years old

Sigmoid colon cancer and diabetes

Administration of the Compound A: 40 mg/m²/day and Metformin Hydrochloride

(Patients 2) Male 57 years old

Colon cancer and diabetes

Administration of the Compound A: 905 mg/m²/day, pioglitazone hydrochloride and Metformin Hydrochloride

(Administration Method and Period)

In addition to already prescribed drugs (pioglitazone hydrochloride or Metformin Hydrochloride) as basic treatment, the Compound A with defined dose for each patient had been intravenously administered for 7 continuous days followed by a 7-day rest period. This 14 day period with a 7-day infusion period and 7-day rest period is defined as one cycle. Basic treatment for diabetes had been continuously performed during this period. During the administration period (first, fifth and eighth day of each cycle) or after the administration, blood glucose level was measured.

FIGS. 1 and 2 show the blood glucose level of each patient. As shown in the Figures, the blood glucose level of each patient was decreased by concomitant administration of Compound A with the known drugs for diabetes (Metformin Hydrochloride alone, or pioglitazone hydrochloride and Metformin hydrochloride). This effect was observed independent of the dose level of Compound A.

No adverse effects of concomitant use of the Compound A were observed.

As shown above, blood glucose level of patients with type II diabetes whose blood glucose level was not enough controlled by any known drugs for type II diabetes alone, were significantly decreased by concomitant use of Compound A.

What is claimed is:

1. An alpha helix mimetic β-catenin inhibitor compound for the treatment of a diabetic condition.

2. A method of treatment for a diabetic condition, comprising administering an effective amount of the compound of claim 1 to a patient in need thereof.

3. The method of claim 2, wherein the condition is Type I diabetes.

4. The method of claim 2, wherein the condition is Type II diabetes.

5. The method of claim 2, wherein the condition is diabetic retinopathy.

6. The method of claim 2, wherein the condition is gestational diabetes.

7. A pharmaceutical composition comprising the compound of claim 1.
